SEQUENCE LISTING (5 sequences)

SEQ ID NO: 1 - DNA, 2010 bp, Homo sapiens
  source 1..2010: mol_type = genomic DNA
atgatgcggctgcgaggctcggggatgctgcgggacctgctcctgcggtcgcccgccggcgtgagcgcgactctgcggcgggcacagcccttggtcaccctgtgccggcgtccccgaggcgggggacggccggccgcgggcccggctgccgccgcgcgactccacccgtggtggggcgggggcggctggccggcggagcccctcgcgcggggcctgtccagctctccttcggagatcttgcaggagctgggcaaggggagcacgcatccgcagcccggggtgtcgccacccgctgccccggcggcgcccggccccaaggacggccccggggagacggacgcgtttggcaacagcgagggcaaagagctggtggcctcaggtgaaaataaaataaaacagggtctgttacctagcttggaagatttgctgttctatacaattgctgaaggacaagagaaaatacctgttcataaatttattacagcactcaaatctacaggattgcgaacgtctgatcccaggttgaaagagtgtatggatatgttaagattaactcttcaaacaacatcagatggtgtcatgctagacaaagatctttttaaaaaatgtgttcagagcaacattgttttgttgacacaagcatttagaagaaagtttgtgattcctgactttatgtcttttacctcacacattgatgagttatatgaaagtgctaaaaagcagtctggaggaaaggttgcagattatattcctcaactggccaaattcagtcccgatttgtggggtgtgtctgtttgtacagtagatggacagaggcattctactggagataccaaagttcccttctgtcttcagtcctgtgtaaaacctttgaaatatgccattgctgttaatgatcttggaactgaatatgtgcatcgatatgttggaaaagagccgagtggactaagattcaacaaactatttttgaatgaagatgataaaccacataatcctatggtaaatgctggagcaattgttgtgacttcactaataaagcaaggagtaaataatgctgaaaaatttgactatgtcatgcagtttttgaataagatggctggtaatgaatatgttggattcagtaatgcaacgtttcagtctgaaagagaaagtggagatcgaaattttgcaataggatattacttaaaagaaaagaagtgttttccagaaggcacagacatggttggtatattagacttctacttccagctgtgctccattgaagtgacttgtgaatcagccagtgtgatggctgcgacactggctaatggtggtttctgcccaattactggtgaaagagtactgagccctgaagcagttcgaaatacattgagtttgatgcattcctgtggcatgtatgacttctcagggcagtttgctttccatgttggtcttcctgcaaaatctggagttgctgggggcattcttttagttgtccccaatgttatgggtatgatgtgctggtctcctcctctggataagatgggcaacagtgttaagggaattcacttttgtcacgatcttgtttctctgtgtaatttccataactatgataatttgagacactttgcaaaaaaacttgatcctcgaagagaaggtggtgatcaaagggtaaagtcagtgataaatcttttgtttgctgcatatactggagatgtgtctgcacttcgaagatttgctttgtcagctatggacatggaacagcgggactatgattctagaacagcactccatgtagctgctgcagagggtcatgttgaagttgttaaatttttgctggaagcctgcaaagtaaaccctttccccaaggacaggtggaataacactcccatggatgaagcactgcactttggacaccatgatgtatttaaaattctccaagaataccaagtccagtacacacctcaaggagattctgacaacgggaaggaaaatcaaaccgtccataagaatcttgatggattgttgtaa

SEQ ID NO: 2 - DNA, 1797 bp, Homo sapiens
  source 1..1797: mol_type = genomic DNA
atgatgcggctgcgaggctcggggatgctgcgggacctgctcctgcggtcgcccgccggcgtgagcgcgactctgcggcgggcacagcccttggtcaccctgtgccggcgtccccgaggcgggggacggccggccgcgggcccggctgccgccgcgcgactccacccgtggtggggcgggggcggctggccggcggagcccctcgcgcggggcctgtccagctctccttcggagatcttgcaggagctgggcaaggggagcacgcatccgcagcccggggtgtcgccacccgctgccccggcggcgcccggccccaaggacggccccggggagacggacgcgtttggcaacagcgagggcaaagagctggtggcctcaggtgaaaataaaataaaacagggtctgttacctagcttggaagatttgctgttctatacaattgctgaaggacaagagaaaatacctgttcataaatttattacagcactcaaatctacaggattgcgaacgtctgatcccaggttgaaagagtgtatggatatgttaagattaactcttcaaacaacatcagatggtgtcatgctagacaaagatctttttaaaaaatgtgttcagagcaacattgttttgttgacacaagcatttagaagaaagtttgtgattcctgactttatgtcttttacctcacacattgatgagttatatgaaagtgctaaaaagcagtctggaggaaaggttgcagattatattcctcaactggccaaattcagtcccgatttgtggggtgtgtctgtttgtacagtagatggacagaggcattctactggagataccaaagttcccttctgtcttcagtcctgtgtaaaacctttgaaatatgccattgctgttaatgatcttggaactgaatatgtgcatcgatatgttggaaaagagccgagtggactaagattcaacaaactatttttgaatgaagatgataaaccacataatcctatggtaaatgctggagcaattgttgtgacttcactaataaagcaaggagtaaataatgctgaaaaatttgactatgtcatgcagtttttgaataagatggctggtaatgaatatgttggattcagtaatgcaacgtttcagtctgaaagagaaagtggagatcgaaattttgcaataggatattacttaaaagaaaagaagtgttttccagaaggcacagacatggttggtatattagacttctacttccagctgtgctccattgaagtgacttgtgaatcagccagtgtgatggctgcgacactggctaatggtggtttctgcccaattactggtgaaagagtactgagccctgaagcagttcgaaatacattgagtttgatgcattcctgtggcatgtatgacttctcagggcagtttgctttccatgttggtcttcctgcaaaatctggagttgctgggggcattcttttagttgtccccaatgttatgggtatgatgtgctggtctcctcctctggataagatgggcaacagtgttaagggaattcacttttgtcacgatcttgtttctctgtgtaatttccataactatgataatttgagacactttgcaaaaaaacttgatcctcgaagagaaggtggtgatcaaaggcattcctttggaccattggactatgaaagtctccaacaagaacttgctttaaaagagacagtatggaaaaaagtgtcacctgagtcaaatgaggacatctctacaactgtagtatatagaatggaaagtctgggagagaaaagctaa

SEQ ID NO: 3 - DNA, 1602 bp, Homo sapiens
  source 1..1602: mol_type = genomic DNA
atggcttttgcaaatctgcggaaagtgctcatcagtgacagcctggacccttgctgccggaagatcttgcaagatggagggctgcaggtggtggaaaagcagaaccttagcaaagaggagctgatagcggagctgcaggactgtgaaggccttattgttcgctctgccaccaaggtgaccgctgatgtcatcaacgcagctgagaaactccaggtggtgggcagggctggcacaggtgtggacaatgtggatctggaggccgcaacaaggaagggcatcttggttatgaacacccccaatgggaacagcctcagtgccgcagaactcacttgtggaatgatcatgtgcctggccaggcagattccccaggcgacggcttcgatgaaggacggcaaatgggagcggaagaagttcatgggaacagagctgaatggaaagaccctgggaattcttggcctgggcaggattgggagagaggtagctacccggatgcagtcctttgggatgaagactatagggtatgaccccatcatttccccagaggtctcggcctcctttggtgttcagcagctgcccctggaggagatctggcctctctgtgatttcatcactgtgcacactcctctcctgccctccacgacaggcttgctgaatgacaacacctttgcccagtgcaagaagggggtgcgtgtggtgaactgtgcccgtggagggatcgtggacgaaggcgccctgctccgggccctgcagtctggccagtgtgccggggctgcactggacgtgtttacggaagagccgccacgggaccgggccttggtggaccatgagaatgtcatcagctgtccccacctgggtgccagcaccaaggaggctcagagccgctgtggggaggaaattgctgttcagttcgtggacatggtgaaggggaaatctctcacgggggttgtgaatgcccaggcccttaccagtgccttctctccacacaccaagccttggattggtctggcagaagctctggggacactgatgcgagcctgggctgggtcccccaaagggaccatccaggtgataacacagggaacatccctgaagaatgctgggaactgcctaagccccgcagtcattgtcggcctcctgaaagaggcttccaagcaggcggatgtgaacttggtgaacgctaagctgctggtgaaagaggctggcctcaatgtcaccacctcccacagccctgctgcaccaggggagcaaggcttcggggaatgcctcctggccgtggccctggcaggcgccccttaccaggctgtgggcttggtccaaggcactacacctgtactgcaggggctcaatggagctgtcttcaggccagaagtgcctctccgcagggacctgcccctgctcctattccggactcagacctctgaccctgcaatgctgcctaccatgattggcctcctggcagaggcaggcgtgcggctgctgtcctaccagacttcactggtgtcagatggggagacctggcacgtcatgggcatctcctccttgctgcccagcctggaagcgtggaagcagcatgtgactgaagccttccagttccacttctaa

SEQ ID NO: 4 - DNA, 1485 bp, Homo sapiens
  source 1..1485: mol_type = genomic DNA
atgctgtacttctctttgttttgggcggctcggcctctgcagagatgtgggcagctggtcaggatggccattcgggctcagcacagcaacgcagcccagactcagactggggaagcaaacaggggctggacaggccaggagagcctgtcggacagtgatcctgagatgtgggagttgctgcagagggagaaggacaggcagtgtcgtggcctggagctcattgcctcagagaacttctgcagccgagctgcgctggaggccctggggtcctgtctgaacaacaagtactcggagggttatcctggcaagagatactatgggggagcagaggtggtggatgaaattgagctgctgtgccagcgccgggccttggaagcctttgacctggatcctgcacagtggggagtcaatgtccagccctactccgggtccccagccaacctggccgtctacacagcccttctgcaacctcacgaccggatcatggggctggacctgcccgatgggggccatctcacccacggctacatgtctgacgtcaagcggatatcagccacgtccatcttcttcgagtctatgccctataagctcaacctggcactgactgctcgacttttccggccacggctcatcatagctggcaccagcgcctatgctcgcctcattgactacgcccgcatgagagaggtgtgtgatgaagtcaaagcacacctgctggcagacatggcccacatcagtggcctggtggctgccaaggtgattccctcgcctttcaagcacgcggacatcgtcaccaccactactcacaagactcttcgaggggccaggtcagggctcatcttctaccggaaaggggtgaaggctgtggaccccaagactggccgggagatcccttacacatttgaggaccgaatcaactttgccgtgttcccatccctgcaggggggcccccacaatcatgccattgctgcagtagctgtggccctaaagcaggcctgcacccccatgttccgggagtactccctgcaggttctgaagaatgctcgggccatggcagatgccctgctagagcgaggctactcactggtatcaggtggtactgacaaccacctggtgctggtggacctgcggcccaagggcctggatggagctcgggctgagcgggtgctagagcttgtatccatcactgccaacaagaacacctgtcctggagaccgaagtgccatcacaccgggcggcctgcggcttggggccccagccttaacttctcgacagttccgtgaggatgacttccggagagttgtggactttatagatgaaggggtcaacattggcttagaggtgaagagcaagactgccaagctccaggatttcaaatccttcctgcttaaggactcagaaacaagtcagcgtctggccaacctcaggcaacgggtggagcagtttgccagggccttccccatgcctggttttgatgagcattga

SEQ ID NO: 5 - DNA, 1053 bp, Homo sapiens
  source 1..1053: mol_type = genomic DNA
atggctgcgacttctctaatgtctgctttggctgcccggctgctgcagcccgcgcacagctgctcccttcgccttcgccctttccacctcgcggcagttcgaaatgaagctgttgtcatttctggaaggaaactggcccagcagatcaagcaggaagtgcggcaggaggtagaagagtgggtggcctcaggcaacaaacggccacacctgagtgtgatcctggttggcgagaatcctgcaagtcactcctatgtcctcaacaaaaccagggcagctgcagttgtgggaatcaacagtgagacaattatgaaaccagcttcaatttcagaggaagaattgttgaatttaatcaataaactgaataatgatgataatgtagatggcctccttgttcagttgcctcttccagagcatattgatgagagaaggatctgcaatgctgtttctccagacaaggatgttgatggctttcatgtaattaatgtaggacgaatgtgtttggatcagtattccatgttaccggctactccatggggtgtgtgggaaataatcaagcgaactggcattccaaccctagggaagaatgtggttgtggctggaaggtcaaaaaacgttggaatgcccattgcaatgttactgcacacagatggggcgcatgaacgtcccggaggtgatgccactgttacaatatctcatcgatatactcccaaagagcagttgaagaaacatacaattcttgcagatattgtaatatctgctgcaggtattccaaatctgatcacagcagatatgatcaaggaaggagcagcagtcattgatgtgggaataaatagagttcacgatcctgtaactgccaaacccaagttggttggagatgtggattttgaaggagtcagacaaaaagctgggtatatcactccagttcctggaggtgttggccccatgacagtggcaatgctaatgaagaataccattattgctgcaaaaaaggtgctgaggcttgaagagcgagaagtgctgaagtctaaagagcttggggtagccactaattaa

(19) United States
(12) Patent Application Publication
(10) Pub. No.: US 2024/0238287 A1
(43) Pub. Date: Jul. 18, 2024

(54) PHARMACEUTICAL COMPOSITION FOR PREVENTING OR TREATING CANCER

(71) Applicants: VeraVerse Co., Ltd., Seoul (KR); INDUSTRY-ACADEMIC COOPERATION FOUNDATION, YONSEI UNIVERSITY, Seoul (KR)

(72) Inventors: Jae-Ho CHEONG, Seoul (KR); Jae-woo KIM, Seoul (KR); Sungsoon FANG, Yongin-si Gyeonggi-do (KR); Bo Kyung YOON, Seoul (KR); Hyeonhui KIM, Seoul (KR)

(73) Assignees: VeraVerse Co., Ltd., Seoul (KR); INDUSTRY-ACADEMIC COOPERATION FOUNDATION, YONSEI UNIVERSITY, Seoul (KR)

(21) Appl. No.: 18/399,675

(22) Filed: Dec. 28, 2023

Related U.S. Application Data
(63) Continuation-in-part of application No. PCT/KR2022/009183, filed on Jun. 28, 2022.

(30) Foreign Application Priority Data
Jun. 28, 2021 (KR) 10-2021-0083887

(51) Int. Cl. A61K 31/501; A61K 31/496; A61P 35/00; C12Q 1/6886

(52) U.S. Cl. CPC A61K 31/501; A61K 31/496; A61P 35/00; C12Q 1/6886; C12Q 2600/106; C12Q 2600/158

(57) ABSTRACT

The present invention relates to a method for prevention, alleviation, or treatment of cancer patients, especially epithelial mesenchymal transition (EMP)-subtype cancer patients. If PHGDH, SHMT and MTHFD2 inhibitors are co-administered to patients with cancer in which the expression level of a glutaminase (GLS) gene or a protein encoded thereby has increased, 1C metabolism is more effectively inhibited such that there is a synergistic effect on the inhibition of cancer cell proliferation in patients with refractory cancer that is difficult to treat because of recurrence, metastasis, and anticancer drug resistance, thereby enabling cancer to be very effectively treated. In addition, the expression level of the GLS gene or protein encoded thereby is measured so that information related to customized treatment methods from initial stages is provided to each patient and the success of treatment can be increased.

### CROSS REFERENCE TO RELATED APPLICATIONS

This application is a continuation-in-part of PCT/KR2022/009183 filed Jun. 28, 2022, which claims priority to the Korean Patent Application No. 10-2021-0083887 filed Jun. 28, 2021, the contents of each of which are incorporated herein by reference in entirety.

### REFERENCE TO AN ELECTRONIC SEQUENCE LISTING

The contents of the electronic sequence listing (SequenceListing.XML; Size: 13 kB; and Date of Creation: Dec. 28, 2023) is herein incorporated by reference in its entirety.

### FIELD OF THE DISCLOSURE

The present invention relates to a pharmaceutical composition for the prevention or treatment of cancer.

### BACKGROUND

Cancer is one of the incurable diseases that humanity has to solve, and huge amounts of capital are being invested in development to cure it all over the world, and in the case of Korea, it is the number one cause of disease death, with more than 100,000 people diagnosed every year and more than 60,000 deaths.

Carcinogens that cause these cancers include smoking, ultraviolet rays, chemicals, food, and other environmental factors, but it is difficult to develop a treatment due to various causes, and the effects of the treatment vary depending on the site where it occurs. Since the substances currently used as therapeutics are highly toxic and do not selectively eliminate cancer cells, there is an urgent need to develop less toxic and effective anticancer drugs to prevent the occurrence of cancer as well as to treat cancer after it occurs. Despite the rapid advances in cancer diagnosis and treatment over the past decade, the fatality rate from cancer incidence remains high.

In particular, stomach cancer is one of the most common malignancies and is the third leading cause of cancer mortality worldwide. Despite great progress in the treatment of gastric cancer patients, there are still limitations in the treatment of gastric cancer patients due to frequent recurrence and metastasis, as well as drug resistance.

[5]Research is actively underway on the mechanisms related to cancer recurrence, metastasis, and drug resistance, and among them, the cancer stem cell hypothesis is attracting attention. Studies have shown the presence of stem-like cells, which contribute to tumor aggressiveness, metastasis, recurrence, and resistance to chemotherapy.

### SUMMARY

The purpose of the present invention is to provide a pharmaceutical composition for the prevention, improvement or treatment of cancer.

Another purpose of the present invention is to provide a method of providing information on treatment options for patients with epithelial mesenchymal transition (EMT) subtype cancer.

However, the technical tasks that the present invention seeks to accomplish are not limited to the tasks mentioned above, and other tasks not mentioned can be clearly understood by persons with ordinary knowledge in the industry from the following descriptions.

One embodiment of the present invention provides a pharmaceutical composition for the prevention or treatment of cancer.

In an embodiment of the present invention, the pharmaceutical composition comprises a compound-capable of inhibiting the expression of a GLS gene, or a pharmaceutically acceptable salt thereof; and an agent capable of inhibiting the expression of any one gene selected from the group consisting of phosphoglycerate dehydrogenase (PHGDH) gene, an Serinehydroxymethyltransferase (SHMT) gene, and an methenyltetrahydrofolatedehydrogenase 2 (NADP+dependent) or methenyltetrahydrofolatecyclohydrolase gene (MTHFD2 gene) or a pharmaceutically acceptable salt thereof as an active ingredient.

In another embodiment of the present invention, the pharmaceutical composition comprises a composition capable of inhibiting the function of a protein encoded by the GLS gene, or a pharmaceutically acceptable salt thereof; and an agent capable of inhibiting the function of a protein encoded by any one gene selected from the group consisting of the PHGDH gene, the SHMT gene, and the MTHFD2 gene, or a pharmaceutically acceptable salt thereof as an active ingredient.

### BRIEF DESCRIPTION OF THE DRAWINGS

FIG. 1 shows the results of gastric transcriptome analysis in elderly cohort gastric cancer patients according to one embodiment of the present invention.

FIG. 2 shows the results of confirming the expression level of proteins encoded by the GLS (glutaminase) gene in intestinal subtype cell lines (NCIN87 and SNU601) and stem-like subtype cell lines (MKN1 and HS746T) according to one embodiment of the present invention through Western blot analysis.

FIG. 3 shows the results of genomic analysis performed on intestinal subtype organoids (GA326) and stem-like subtype organoids (GA077) according to one embodiment of the present invention.

FIGS. 4 to 7 show the results of checking the proliferation level of the cell line according to the presence or absence of glutamine (FIG. 4) and the concentration of DON, which is an analogue of glutamine according to one embodiment of the present invention (FIG. 5), and the result of confirming the proliferation level of the cell line according to the concentration of the GLS inhibitors CB839 (FIG. 6) and BPTES (FIG. 7).

FIG. 8 shows the result of confirming the change in the size of the stem-like subform organoid by treatment with the GLS inhibitor CB839 alone according to one embodiment of the present invention.

FIGS. 9 to 11 show the results of checking the proliferation level of stem-like subtype cell lines with the presence or absence of glutamine and combination treatment with PHGDH (FIG. 9), SHMT (FIG. 10) or MTFHD2 (FIG. 11) inhibitors according to one embodiment of the present invention.

FIG. 12 shows the result of confirming the change in the size of the stem-like subform organoid by the combination treatment of the GLS inhibitor (CB839) and NCT503 according to one embodiment of the present invention.

FIGS. 13, 14, and 15 show the effect of GLS inhibition activating ATF4/CEBPB-mediated transcriptional network in EMT signature-enriched clusters in patient-derived cancer organoids.

FIG. 13 shows diagram to explain the process in vivo experiment with vehicle, BPTES (12.5 mg/kg), NCT503 (40 mg/kg), or combination of BPTES and NCT503.

FIG. 14 shows representative picture of mice (n=5) with tumor volume measured via in vivo optical imaging system. Total radiant efficiency (p/sec/cm2/sr/μW/cm2) was measured in peritoneal area.

FIG. 15 shows total radiant efficiency was compared in every group (n=7) before (D7) and after (D23) three cycles of BPTES/NCT503 injection.

### DETAILED DESCRIPTION

The “GLS gene” of the present invention is a gene encoding k-type mitochondrial glutaminase, which catalyzes the hydrolysis of glutamine into glutamate and ammonia. In particular, cancer cells have been reported to overexpress the GLS gene in order to obtain a large amount of energy source and a source necessary for the synthesis of fatty acids, thereby degrading glutamine. For the purposes of the present invention, cancer patients may have overexpression of the GLS gene, which may be characterized by inhibition of proliferation or cell death by agents that can inhibit the expression of the GLS gene or the function of the protein encoded thereby, but is not limited thereto.

The GLS gene of the present invention may consist of a base sequence represented by SEQ ID NO: 1 or SEQ ID NO: 2, but is not limited thereto.

A compound capable of inhibiting the function of proteins encoded by the GLS gene of the present invention may be compounds labeled with Chemical Formula 1 (CAS No. 1439399-58-2) or Chemical Formula 2 (CAS no. 314045-39-1), but are not limited to:

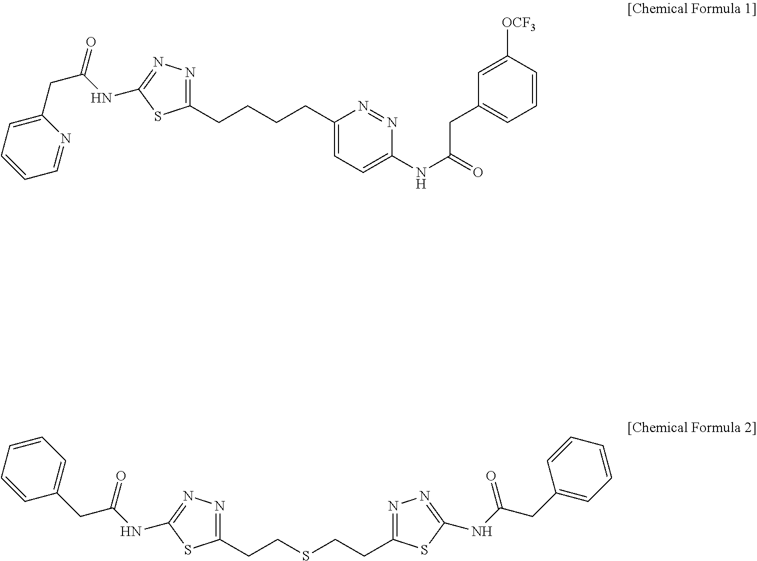 [SMILES: O=C(Cc1cccc(OC(F)(F)F)c1)Nc1ccc(CCCCc2nnc(NC(=O)Cc3ccccn3)s2)nn1 ; O=C(Cc1ccccc1)Nc1nnc(CCSCCc2nnc(NC(=O)Cc3ccccc3)s2)s1]

The “PHGDH gene” of the present invention is a gene encoding an enzyme involved in the early stages of L-serine synthesis in animal cells.

The PHGDH gene of the present invention may consist of a base sequence represented by SEQ ID NO: 3, but is not limited thereto.

The “SHMT gene” of the present invention is a gene encoding an enzyme that catalyzes the reversible conversion of serine and tetrahydrofolate to glycine and 5,10-methylentetrahydrofolate.

The SHMT gene of the present invention may consist of a base sequence represented by SEQ ID NO: 4, but is not limited thereto.

The “MTHFD2 gene” of the present invention is a gene encoding an enzyme having methylenetetrahydrofolate dihydrogenase or methenyltetrahydrofolatecyclohydrolase activity.

The MTHFD2 gene of the present invention may consist of a base sequence represented by SEQ ID NO: 5, but is not limited thereto.

An example of compounds capable of inhibiting the function of proteins encoded by the PHGDH gene of the present invention may be compounds denoted by the following Chemical Formula 3 (CAS No. 1916571-90-8), but are not limited to:

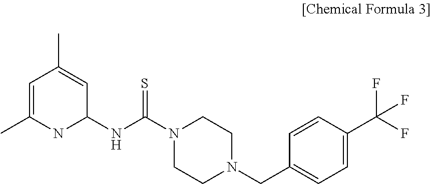

An example of compounds capable of inhibiting the function of proteins encoded by the MTHFD2 gene of the present invention may be compounds denoted by the following Chemical Formula 4 (CAS No. 2227149-22-4), but are not limited to:

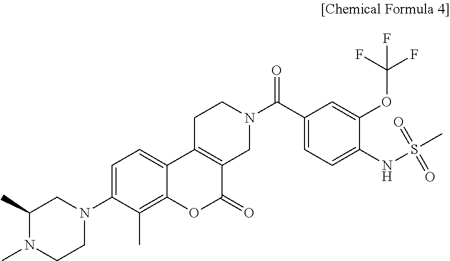

A compound capable of inhibiting the function of proteins encoded by the SHMT gene of the present invention may be compounds denoted by the following Chemical Formula 5 (CAS No. 2146095-85-2), but are not limited to:

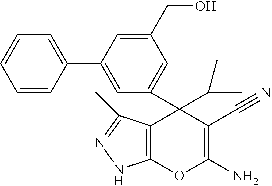 [SMILES: Cc1n[nH]c2c1C(c1cc(CO)cc(-c3ccccc3)c1)(C(C)C)C(C#N)=C(N)O2]

For the purpose of the present invention, the PHGDH, SHMT and MTHFD2 genes and the proteins encoded thereby have increased expression in cancer patients together with the GLS gene and the proteins encoded thereby, so that when administered in combination with a drug that can inhibit the expression of the GLS gene or the function of the protein encoded thereby, it is possible to exert a remarkable synergistic effect on proliferation inhibition and death of cancer cells in cancer patients.

The “pharmaceutically acceptable salt” of the present invention is a salt that is generally considered by a person in the art to be suitable for medical application (e.g., because such salt is not harmful to the subject that can be treated with the salt), or a salt that causes an acceptable side effect within the respective treatment. Generally, the above pharmaceutically acceptable salts are those that are considered acceptable by regulatory authorities such as the U.S. Food and Drug Administration (FDA), the European Medicines Agency (EMA), or the Pharmaceuticals and Medical Devices Agency (PMDA) of the Japanese Ministry of Health and Welfare.

The pharmaceutical composition of the present invention, in each case, the person skilled in the art can easily determine whether a particular compound according to the present invention or a physiologically functional derivative thereof can form a salt, that is, whether the substance corresponding to the inhibitor under the present invention or the physiologically functional derivative thereof, e.g., has an electric charge such as an amino group, a carboxylic acid group, etc.

If the inhibitable Chemical Formulation of the present invention is a compound, the exemplary salt of the compound is an acid adjunct salt or a salt with a base, in particular a pharmaceutically acceptable inorganic and organic acid adjunct salt and a salt with a base commonly used in pharmaceuticals, which is water-insoluble or a particularly water-soluble acid adjunct. Depending on the substituent of the compound, salts with bases may also be suitable. Acid adjunct salt may be formed, for example, by mixing a solution of a compound of the present invention with a solution of a pharmacologically acceptable acid, such as hydrochloric acid, sulfuric acid, fumaric acid, maleic acid, succinic acid, acetic acid, benzoic acid, citric acid, tartaric acid, carbonic acid or phosphoric acid. Likewise, pharmaceutically acceptable base adjunct salts are alkaline metal salts (e.g., sodium or potassium salts); alkaline earth metal salts (for example, calcium or magnesium salts); and salts formed with suitable organic ligands (e.g., ammonium, quaternary ammonium, and amine cations formed using opposite anions such as halides, hydroxides, carboxylates, sulfates, phosphates, nitrates, alkyl sulfonates, and aryl sulfonates). Illustrative examples of pharmaceutically acceptable salts are: acetate, adifate, alginate, arginate, ascorbate, aspartate, benzenesulfonate, benzoate, bicarbonate, bisulfate, biotrate, borate, bromide, butyrate, calcium edetate, camporate, camphosulfonate, camsylate, carbonate, chloride, citrate, digluconate, dihydrochloride, dodecyl sulfate, edetate, edysylate, ethane Sulfonate, Formmate, Fumarate, Galactate, Galacturonate, Gluconate, Glutamate, Glycerophosphate, Hemisulfate, Heptanoate, Hexylesorinate, Hydrobromide, Hydrochloride, Hydroiodide, 2-Hydroxy-Ethane Sulfonate, Hydroxynaphthoate, lodide, Isobutyrate, Isothionate, Lactate, Laurerate, Lauryl Sulfate, Malate, maleate, malonate, mandelate, methanesulfonate (mesylate), methyl sulfate, 2-naphthalene sulfonate, nicotinate, nitrate, oleate, oxalate, palmitate, pantothenate, pectinate, persulfate, 3-phenylpropionate, phosphate/diphosphate, phthalates, picrate, pibalate, polygalacturonate, propionate, salicylate, stearate, sulfate, suberate, succinate, tannate, tartrate, tosylate, undecanoate, valerate, etc., but is not limited to thereto.

The a compound that can inhibit the expression of the aforementioned genes of the present invention are compounds; It may be, but is not limited to, any one selected from a group of miRNAs, siRNAs, shRNAs, and antisense oligonucleotides that bind specifically to the mRNA of the gene.

The Chemical Formulation capable of inhibiting the function of the protein of the present invention may be one of the compounds, inverse agonists, antagonists, and any one selected from the group consisting of antibodies or aptamers that can bind specifically to the proteins.

The “inverse agonist” or “antagonist” of the present invention means a molecule that can directly or indirectly reduce the biological activity of the receptor, including, but not limited to, a molecule that, when used in conjunction with a ligand of the receptor, may reduce the action of the ligand.

The “antibody” of the present invention means a protein-like molecule which can bind specifically to the antigenic site of a protein or peptide molecule, and such an antibody is obtained by cloning each gene to the expression vector according to the usual method to obtain the protein encoded by the marker gene, and can be prepared by the usual method from the obtained protein.

The “aptamer” of the present invention means a nucleic acid molecule having binding activity to a predetermined target molecule. The aptamer may be RNA, DNA, modified nucleic acids, or a mixture thereof, and may be in the form of a straight chain or a loop, and it is generally known that the shorter the sequence of the nucleotides constituting the aptamer, with easier chemical synthesis and mass production, better cost advantages, cleaner Chemical Formula, better stability in vivo, and lower toxicity.

The above cancers of the present invention include gastric cancer, thyroid cancer, parathyroid cancer, ovarian cancer, colorectal cancer, pancreatic cancer, liver cancer, breast cancer, cervical cancer, lung cancer, non-small cell lung cancer, prostate cancer, gallbladder cancer, biliary tract cancer, non-Hodgkin lymphoma, Hodgkin lymphoma, blood cancer, bladder cancer, kidney cancer, melanoma, colon cancer, bone cancer, skin cancer, head cancer, uterine cancer, rectal cancer, brain tumor, cancer, fallopian tube carcinoma, endometrial carcinoma, vaginal cancer, scrotal carcinoma, esophageal cancer, small intestine cancer, endocrine gland cancer, adrenal cancer, soft tissue sarcoma, urethral cancer, penile cancer, ureteral cancer, renal cell carcinoma, renal pelvic carcinoma, CNS central nervous system tumor, It may be any one selected from a group consisting of primary CNS lymphoma, spinal cord tumor, brainstem glioma, and pituitary adenoma, for example, stomach cancer, but not limited to.

The cancer of the present invention may be an incurable cancer, which is a metastasis, recurrence, and drug-resistant cancer, but is not limited to.

The cancer in the present invention may be an epithelial mesenchymal transition (EMT) subtype, but is not limited to.

The “EMT molecular subtype” of the present invention is a subtype in which there is a process of transformation of epithelial cells into mesenchymal cells, and it is a mutation process in which epithelial cells lose their appearance and have the characteristics of mesenchymal cells, and it is known to be an important process in the development of individual formation, and refers to a molecular subtype in which cancer cell growth, drug resistance, infiltration and metastasis may occur.

The composition of the present invention may further include anticancer drugs.

The anticancer drugs of the present invention consist of nitrogen mustard, imatinib, oxaliplatin, rituximab, erlotinib, neratinib, lapatinib, zefitinib, bandantanib, nirotinib, semasanib, bosutinib, axitinib, cediranib, lestaurtinib, trastuzumab, gefitinib, bortezomib, sunitinib, carboplatin, sorafenib, bevacizumab, cisplatin, cetuximab, biscumalbum, asparaginase, tretinoin, hydroxycarbamide, dasatinib, estramostine, gemtuzumapozogamycin, ibritumomab tucetan, heptaplatin, methylaminolevulinic acid, amsacrine, alemtuzumab, procarbazine, alprostadil, holmium nitrate chitosan, gemcitabine, doxyfluridine, pemetrexed, tegapur, capecitabine, zimeracin, oterasil, azacitidine, methotrexate, uracil, cytarabine, Fluorouracil, fludagovine, enositabine, flutamide, kepesitabine, decitabine, mercaptopurine, thioguanine, cladribine, carmofer, ralitrexed, docetaxel, paclitaxel, irinotecan, belotecan, topotecan, vinorellevine, etoposide, vincristine, vinblastine, tenifoside, doxorubicin, idarubicin, epirubicin, mitoxantrone, mitomycin, blelomycin, daunosecinRuvicin, dactinomycin, pyrarubicin, aclarubicin, pepromycin, temsirolimus, temozolomide, busulfan, iphosphamide, cyclophosphamide, melparan, altretmine, dacarbazine, thiotepa, nimustine, chlorambucil, mitolactol, leucovorin, tretonin, exmestane, aminoglutesimide, anagrelide, olaparib, nabelvin, padrazole, tamoxifen, toremifene, testosterone, anastrozole, One or more species selected from the group consisting of letrozole, borozole, bicalutamide, lomustine, borinostat, entinoste, penformin, metformin, talazoparib, and carmustine may be used, but not limited to.

The above “prevention” in the present invention means any act that inhibits or delays the onset of a disease or pathology. For the purposes of the present invention, the composition is meant to delay the onset of cancer, especially EMT molecular subtype cancer, or to inhibit its onset.

The “cure” of the present invention means any act that delays, stops, or reverses the progression of disease or disease, and for the purposes of the present invention the composition means to stop, reduce, mitigate, eliminate or reverse the progression of cancer, especially EMT molecular subtype cancer.

The pharmaceutical composition of the present invention may be characterized as being in the form of capsules, tablets, granules, injections, ointments, powders, or beverages, and the pharmaceutical composition may be characterized by human subjects.

The pharmaceutical composition of the present invention is not limited to these, but each may be Chemical Formulated and used in the form of an oral Chemical Formulation, topical agent, suppository, and sterile injection solution such as an acid, granule, capsule, tablet, aqueous suspension, etc., according to the usual method. The pharmaceutical composition of the present invention may include a pharmacologically acceptable carrier. The pharmacically acceptable carrier can be used as a binder, a synovage, a disintegrating agent, an excipient, a solubilizer, a dispersing agent, a stabilizer, a suspension agent, a color, a fragrance, etc., and in the case of an injection, a buffer, a preservative, a non-monetizing agent, a solubilizing agent, an isotonic agent, a stabilizer, etc., can be used, and in the case of topical administration, a base, excipient, lubricant, preservative, etc. can be used.

The Chemical Formulation of the pharmaceutical composition of the present invention may be prepared in a variety of ways by mixing with a pharmaceutically acceptable carrier as described above. For example, when administered orally, it can be prepared in the form of tablets, troki, capsules, elixirs, suspensions, syrups, wafers, etc., and in the case of injections, it can be prepared in the form of unit-dose ampoules or multi-doses. It can be Chemical Formulated into others, solutions, suspensions, tablets, capsules, extended-release Chemical Formulations, etc.

Examples of carriers, excipients, and diluents suitable for the Chemical Formulation of the present invention include lactose, dextrose, sucrose, sorbitol, mannitol, xylitol, erythritol, maltitol, starch, gum acacia, alginate, gelatin, calcium phosphate, calcium silicate, cellulose, methylcellulose, microcrystalline cellulose, polyvinylpyrrolidone, water, methyl hydroxybenzoate, propyl hydroxybenzoate, talc, magnesium stearate or mineral oil. In addition, it may additionally contain fillers, antiflocculants, lubricants, wetting agents, fragrances, emulsifiers, preservatives, etc.

The route of administration of the pharmaceutical composition of the present invention is not limited to them, but includes oral, intravenous, intramuscular, intraarterial, intramedullary, intrathecal, intracardiac, percutaneous, subcutaneous, intraperitoneal, intranasal, enteral, local, sublingual or rectal. Oral or parenteral dropping is preferable. In the present invention, the “parenteral” includes subcutaneous, intradermal, intravenous, intramuscular, intraarticular, bursal, sternal, intrathecal, intralesional, and intracranial injection or injection techniques. In addition, the pharmaceutical composition can be administered in the form of suppositories for rectal administration.

The pharmaceutical composition of the present invention may vary depending on a number of factors, including the activity of the particular compound used, age, weight, general health, sex, Chemical Formulation, time of administration, route of administration, discharge rate, drug Chemical Formulation, and the severity of the particular disease to be prevented or treated, and the dosage of the pharmaceutical composition depends on the patient's condition, weight, degree of disease, drug form, route and duration of administration, but can be appropriately selected by the person in the art. It can be administered at 0.0001 to 50 mg/kg or 0.001 to 50 mg/kg per day. It can be administered once a day or divided into several doses. The above dosage does not in any way limit the scope of the present invention. The pharmaceutical composition according to the present invention can be Chemical Formulated as a pill, dragee, capsule, liquid, gel, syrup, slurry, suspension.

In another embodiment of the present invention, a Chemical Formulation capable of inhibiting the expression of Glutaminase (GLS) gene in an entity requiring administration, or a pharmaceutically acceptable salt thereof; and a group of phosphoglycerate dehydrogenase (PHGDH) genes, Serine hydroxymethyltransferase (SHMT) genes, and Methylenetetrahydrofolate Dehydrogenase (NADP+ Dependent) 2 or MethenyltetrahydrofolateCyclohydrolase (MTHFD2) genes that can inhibit the expression of any one gene, or a drug containing a pharmaceutically acceptable salt thereof in a pharmaceutically effective dose.

In the method of prevention or treatment of the cancer of the present invention, the GLS, PHGDH, SHMT or MTHFD2 genes, a compound capable of inhibiting the expression of the gene, pharmaceutically acceptable salts and cancers are the same as those described in the composition for the prevention, improvement or treatment of the cancer, and are omitted to avoid excessive complexity of the present specification.

In the present invention, the “dosing” means to provide the object with a prescribed composition of the present invention in any appropriate manner.

In the present invention, the “object” requiring the above dosing may include both mammals and non-mammals. Here, examples of said mammals include humans, non-human primates, e.g., chimpanzees, other ape or monkey species; livestock animals, e.g. cattle, horses, sheep, goats, pigs; domesticated animals, e.g. rabbits, dogs or cats; Laboratory animals, such as rodents, e.g. rats, mice or guinea pigs, may include, but are not limited to. In addition, in the present invention, the examples of the non-mammals may include, but are not limited to, birds or fish.

The Chemical Formulation administered as described above in the present invention is not specifically limited, and may be administered as a solid form, liquid form, or aerosol Chemical Formulation for aspiration, and may be administered as a solid form Chemical Formulation intended to be converted into a liquid form Chemical Formulation for oral or parenteral administration immediately prior to use, e.g., an acid, granule, capsule, tablet, oral Chemical Formulation of an aqueous suspension, topical use, suppositories and sterile injection solution, etc. This is not limited to this.

In addition, in the present invention, at the time of the above administration, a pharmacologically acceptable carrier may be additionally administered along with the Chemical Formulation of the present invention. Here, the pharmaceutically acceptable carrier can be used as a binder, synovitizer, disintegrator, excipient, solubilizer, dispersant, stabilizer, suspension agent, color, fragrance, etc., when administered orally, and in the case of injection, a buffer, preservative, non-monetizer, solubilizer, isotonic agent, stabilizer, etc., a mixture can be used, and in the case of topical administration, a base, excipient, lubricant, preservative, etc. can be used. The Chemical Formulation of the compound of the present invention may be prepared in a variety of ways by mixing with a pharmaceutically acceptable carrier as described above. For example, when administered orally, it can be prepared in the form of tablets, trochi, capsules, elixir, suspension, syrup, wafers, etc., and in the case of injections, it can be prepared in the form of unit-dose ampoules or multi-doses. It can be Chemical Formulated as others, solutions, suspensions, tablets, capsules, extended-release Chemical Formulations, etc.

On the other hand, examples of carriers, excipients, and diluents suitable for Chemical Formulation include lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate or mineral oil. In addition, it may additionally contain fillers, antiflocculants, lubricants, wetting agents, fragrances, emulsifiers, preservatives, etc.

The route of administration of the preparation pursuant to the present invention is but is not limited to these: oral, intravenous, intramuscular, intra-arterial, intramedullary, intradural, intracardiac, transdermal, subcutaneous, intra-abdominal cavity, intranasal, enteral, topical, sublingual or rectal. Oral or parenteral dropping is preferable.

In the present invention, “parenteral” includes subcutaneous, intradermal, intravenous, intramuscular, intraarticular, bursal, sternal, intrathecal, intralesional, and intracranial injection or injection techniques. The pharmaceutical composition of the present invention can also be administered in the form of a suppository for rectal administration.

In the present invention, “pharmaceutically effective amount” refers to a sufficient amount of an agonist to provide a desired biological outcome. The result may be a reduction and/or alleviation of the signs, symptoms or causes of the disease, or any other desirable change in the biological world. For example, the “effective dose” for therapeutic use is the amount of Chemical Formulation disclosed in the present invention that is required to provide a clinically significant reduction in the disease. The appropriate amount of “effective” in any individual case can be determined by a person skilled in the arts using routine experiments. Thus, the expression “effective dose” usually refers to the amount in which the active substance has a therapeutic effect. In the present case, the active substance is a growth inhibitor of cancer cells and a preventive, ameliorating or curative of cancer.

The Chemical Formulation of the present invention may vary depending on several factors, including activity, age, weight, general health, sex, Chemical Formulation, time of administration, route of administration, discharge rate, drug Chemical Formulation, and severity of the specific disease to be prevented or treated, and the dosage of the preparation varies depending on the patient's condition, weight, degree of disease, drug form, route and duration, but can be appropriately selected by the person in the art, and can be administered at 0.0001 to 100 mg/kg or 0.001 to 100 mg/kg per day. It can be administered once a day or divided into several doses. The above dosage does not in any way limit the scope of the present invention. Compounds according to the present invention may be Chemical Formulated as pills, dragees, capsules, liquids, gels, syrups, slurries, suspensions.

The preparation of the present invention can be used alone or in combination with methods such as surgery, radiotherapy, hormone therapy, chemotherapy, and biologic reaction modulators.

In addition, the Chemical Formulation of the present invention can be used in combination with other anticancer drugs, at which time the anticancer drugs include nitrogen mustard, imatinib, oxaliplatin, rituximab, panitumumab, erlotinib, neratinib, lapatinib, zefitinib, bandantanib, nirotinib, semasanib, bosutinib, axitinib, cediranib, lestaurtinib, trastuzumab, gefitinib, bortezomib, susutricanibNitinib, Carboplatin, 5-Fluorouracil (5-FU), Bevacizumab, Cisplatin, Cetuximab, Applibercept, Regorafenib, Biscumalbum, Asparaginase, Tretinoin, Hydroxycarbamide, Dasatinib, Estramostine, GemtuzumabOzogamycin, Ibritumomab Tucetan, Heptaplatin, Methylaminolevulinic Acid, Amsacrine, Alemtuzumab, Procarbazine, Alprostadil, Holmium Nitrate Chitosan, Gemcitabine, Doxyfluridine, Pemetrexed, Tegapur, Capecitabine, Gimeracin, Oteracil, Azacitidine, Methotrexate, Uracil, Cytarabine, Fluorouracil, FludaGabine, Enositabine, Flutamide, Decitabine, Mercaptopurine, Thioguanine, Cladribine, Leucovorin, Carmoper, Raltitrexed, Interferon alfafa-2a, Docetaxel, paclitaxel, irinotecan, belotecan, topotecan, vinorellevine, etoposide, vincristine, vinblastine, tenifoside, doxorubicin, idarubicin, epirubicin, mitoxantron, mitomycin, blelomycin, daunorubicin, dactinomycin, pyrarubicin, aclavivicin, pepromycin, temsirolimus, temozolomide, 5-fluorouracil, adsulfan, iphosphamide, cyclophosphamide, Melparan, Altretmine, Dacarbazine, Thiotepa, Nimustine, Chlorambucil, Mitolactol, Leucovorin, Trettonin, Exmestane, Aminoglutesimide, Anagrelide, Nabelvin, Padrazole, Tamoxifen, Toremifene, Testosterone, Anastrozole, Letrozole, Borozole, Bicalutamide, Lomustine, and Carmustine may be used, but is not limited to.

Another embodiment of the present invention provides a method of providing information on how to treat patients with EMT molecular subtype cancer.

The method of the present invention is to measure the expression level of the GLS gene or the protein encoded thereby in a biological sample isolated from the object individual; and if the expression level of the gene or protein measured above is increased, the Chemical Formulation or a compound that can inhibit the expression of the following genes; or judging by the concomitant administration of a preparation or agent capable of inhibiting the function of proteins encoded by the following genes; Includes:
PHGDH (Phosphoglycerate Dehydrogenase) genes,
SHMT (Serine hydroxymethyltransferase) genes, and
MTHFD2 (Methylenetetrahydrofolate Dehydrogenase (NADP+ Dependent) 2 or MethenyltetrahydrofolateCyclohydrolase) genes.

In the present invention, the “object of interest” means a Chemical Formulation capable of inhibiting GLS gene expression; or an individual whose response to treatment is uncertain due to a drug that can inhibit the function of the protein encoded by it, and who has developed cancer or has a high probability of developing cancer.

The “biological sample” of the present invention means any substance, biological body fluid, tissue, or cell obtained from or derived from an individual, e.g., whole blood, leukocytes, peripheral blood mononuclear cells, white blood buffy coat, plasma, and serum) blood, sputum, tears, mucus, nasal washes, nasal aspirate, breath, urine, semen, saliva, peritoneal washings, pelvic fluids, cystic fluid, meningeal fluid, amniotic fluid, glandular fluid, pancreatic fluid, lymph fluid, pleural fluid, nipple aspirate, bronchial aspirate, synovial fluid, joint aspirate, organ secretions, It may include, but is not limited to, cells, cell extracts, or cerebrospinal fluid.

The method of measuring the expression level of the protein in the present invention may be at least one selected from the group consisting of protein chip analysis, immunoassay, ligand binding assay, MALDI-TOF (matrix assisted laser description/ionization time of flight mass spectrometry) analysis, SELDI-TOF (surface enhanced laser description/ionization time of flight mass spectrometry) analysis, radiation immuno-diffusion, radiation immunodiffusion, an orotate immune diffusion method, roketimmunoelectrophoresis, tissue immunostaining, complement fixation analysis, two-dimensional electrophoresis analysis, liquid chromatography-mass spectrometry (LC-MS), LC-MS/MS (liquid chromatography/mass spectrometry), western blotting and ELISA (enzyme linked immunosorbent assay).

The expression level of the protein of the present invention can be measured using a Chemical Formulation that can measure the expression level of the protein. A Chemical Formulation capable of measuring the expression level of the protein may be at least one selected from a group consisting of an antibody, an oligopeptide, a ligand, a peptide nucleic acid (PNA) and an aptamer that binds specifically to the protein.

The “antibody” of the present invention refers to a substance that specifically binds to an antigen and produces an antigen-antibody reaction. For the purposes of the present invention, an antibody means an antibody that binds specifically to the protein. The antibodies of the present invention include both polyclonal antibodies, monoclonal antibodies, and recombinant antibodies. The antibody can be readily prepared using a technology widely known in the art industry. For example, polyclonal antibodies may be produced by a method widely known in the art industry, which involves the process of injecting the antigen of the protein into an animal and collecting blood from the animal to obtain a serum containing the antibody. These polyclonal antibodies can be manufactured from any animal, such as goats, rabbits, sheep, monkeys, horses, pigs, cows, and dogs. In addition, monoclonal antibodies are widely known in the industry as the hybridoma method; Kohler and Milstein (1976) European Journal of Immunology 6:511-519), or phage antibody library techniques (Clackson et al, Nature, 352:624-628, 1991; Marks et al, J. Mol. Biol., 222:58, 1-597, 1991). Antibodies prepared by the method can be isolated and purified using methods such as gel electrophoresis, dialysis, salt precipitation, ion exchange chromatography, affinity chromatography, etc. In addition, the antibody of the present invention comprises a complete form having two full-length light chains and two full-length heavy chains, as well as a functional fragment of the antibody.

The functional fragment of the antibody of the present invention means a fragment possessing at least an antigen binding function, and includes Fab, F(ab′), F(ab′)2 and Fv.

### Gastric Cancer Treatment

Gastric cancer (GC) is one of the leading causes of cancer-related deaths with high degree of intratumoral heterogeneity.

The invention provides a treatment strategy for chemoresistant GC patients via deciphering metabolic plasticity of Stem-like/Epithelial-to-mesenchymal transition/Mesenchymal-type GC cells. Research and data (YOON B K, et al., PNAS, 2023, Vol. 120, No. 21, e2217826120) provides molecular mechanisms behind the metabolic plasticity of stemness-high GC cells, from chromatin architecture to transcriptional drivers ATF4/CEBPB, and the importance of reactive oxygen species scavenging activity mediated by mitochondrial 1C metabolism. Further understanding of 1C metabolism may lead to development of therapeutic strategies for chemoresistant GC patients, as verified in the patient-derived cancer organoids.

Molecular classification of gastric cancer (GC) identified a subgroup of patients showing chemoresistance and poor prognosis, termed SEM (Stem-like/Epithelial-to-mesenchymal transition/Mesenchymal) type. The study shows that SEM-type GC exhibits a distinct metabolic profile characterized by high glutaminase (GLS) levels. Unexpectedly, SEM-type GC cells are resistant to glutaminolysis inhibition. The study shows that under glutamine starvation, SEM-type GC cells up-regulate the phosphoglycerate dehydrogenase (PHGDH)-mediated mitochondrial folate cycle pathway to produce NADPH as a reactive oxygen species scavenger for survival. This metabolic plasticity is associated with globally open chromatin structure in SEM-type GC cells, with ATF4/CEBPB identified as transcriptional drivers of the PHGDH-driven salvage pathway. Single-nucleus transcriptome analysis of patient-derived SEM-type GC organoids revealed intratumoral heterogeneity, with stemness-high subpopulations displaying high GLS expression, a resistance to GLS inhibition, and ATF4/CEBPB activation. Notably, coinhibition of GLS and PHGDH successfully eliminated stemness-high cancer cells. Together, these results provide insight into the metabolic plasticity of aggressive GC cells and indicates a treatment strategy for chemoresistant GC patients.

Gastric cancer (GC) is a highly heterogeneous adenocarcinoma. The Cancer Genome Atlas (TCGA) project and Asian Cancer Research Group (ACRG) have defined clinically relevant molecular subtypes of GC. The genetically stable subtype from the TCGA project and epithelial-to-mesenchymal transition (EMT) subtype from the ACRG study, although not identical, have a high percentage of diffuse histological types, and present a worse prognosis than intestinal histological types. Studies have subtyped GC patients in the Yonsei cohort into five groups—gastric, inflammatory, intestinal, mixed, and stem-like subtypes—with the stem-like subtype displaying the most aggressive phenotype and resistance to chemotherapy. However, there are currently no targeted treatments available for this aggressive subtype, which studies refer to as SEM (stem-like/EMT/mesenchymal) in this study. The studies shows that a thorough understanding of the molecular characteristics in aggressive SEM-type GC would be helpful.

Glutamine is a ready source of carbon and nitrogen for numerous biosynthetic pathways. Glutamine first undergoes glutaminolysis to become glutamate via the action of glutaminase (GLS). While GLS2 (liver-type) is expressed primarily in the liver, brain, and pancreas, GLS (kidney-type) is broadly expressed in various tissues including cancers with the known importance of GLS in tumorigenesis and cancer cell proliferation, GLS has been widely investigated as a therapeutic target for cancer cells, particularly those in a high mesenchymal state. Indeed, selective inhibitors of GLS are currently being tested in clinical trials in refractory solid tumors. As it was reported, SEM-type GC is chemoresistant while maintaining a high mesenchymal state, raising the potential for GLS to be an effective therapeutic target.

However, cancer cells can adapt their metabolism to survive through activating compensatory metabolic pathways. Furthermore, intratumoral metabolic heterogeneity leads to the cell-to-cell difference in sensitivity to the anticancer drugs within a tumor, resulting in incomplete treatment response and acquired therapeutic resistance. Particularly, cancer stem cells often exhibit chemoresistance due to their quiescent state and slow proliferation rate. As cancer stem cells are known to be critical in the formation and maintenance of liver metastases in aggressive GC, failure in the elimination of cancer stem cells can result in reemergence of the disease. Therefore, there is an urgent, unmet need for a therapeutic regimen that enables simultaneous elimination of each cancer cell subpopulation including cancer stem cells, which display a high degree of plasticity.

The study by YOON et al. in PNAS demonstrates that while SEM-type GC is GLS-positive, it exhibits resistance to GLS inhibition. SEM-type GC cells have a globally open chromatin structure that is accessible to binding with ATF4 and CEBPB, contributing to the activation of genes involved in the PHGDH-driven salvage pathway that controls mitochondrial reactive oxygen species (ROS) in response to GLS inhibition. Single-nucleus transcriptome analysis revealed that GLS-positive clusters in organoids derived from SEM-type GC patients express high levels of cancer stem cell marker CD44 and WNT signaling. The combinatorial inhibition of GLS and PHGDH reduces the survival of stem-like GC. Thus, studies demonstrate the effectiveness of combination therapy targeting chemoresistant stemness-high GC.

PHGDH-Driven Salvage Pathway Inhibits Accumulation of Mitochondrial ROS. Stem cells maintain globally open chromatin to facilitate accessibility of target motifs by transcription factors. To determine SEM-type-specific responses and metabolic robustness during glutamine starvation, the studies conducted an assay for transposase-accessible chromatin using sequencing (ATAC-seq). Differences in chromatin accessibility between the cell lines were apparent in nucleosome-free regions. However, the data shows that only HS746T exhibited a net increase in accessibility upon glutamine starvation. In addition, the number of opening and closing peaks upon glutamine starvation was greater in HS746T than in NCIN87 cells. Regions with increased chromatin accessibility following glutamine starvation were enriched in pathways related to the oxidative stress responses in both cell lines. However, pathways that protect against oxidative stress, such as regulation of oxidative stress-induced cell death and regulation of cellular response to oxidative stress, were only enriched in the newly opened regions of HS746T cells. Metabolic stress, including glutamine starvation, produces ROS, and consecutive activation of PHGDH, SHMT2, and MTHFD2 leads to the production of NADPH to minimize ROS-induced cellular stress. NADPH is an important reducing agent that eliminates cellular ROS and maintains redox homeostasis. As PHGDH was the most up-regulated gene with the lowest adjusted P-value, it was investigated the combinatorial effect of glutamine starvation and PHGDH inhibitor NCT503 in SEM-type GC cells. Indeed, the ratio of reduced glutathione (GSH) to oxidized glutathione (GSSG) decreased when HS746T cells were cultured without glutamine along with the pharmaceutical inhibition of PHGDH. The amount of NADP, the oxidized form of NADPH, was also lowest in PHGDH-inhibited cells with glutamine deficiency. Indeed, cell death induced by cotreatment with CB839 and NCT503 was partially rescued by the addition of the antioxidant N-acetylcysteine (NAC). Mitochondrial ROS estimated by MitoSOX was also the highest with NCT503 treatment and glutamine starvation.

The study also generated an HS746T shPHGDH stable cell line to stably knockdown gene expression of PHGDH and measured oxidative stress in the absence of glutamine. Both oxidative stress and mitochondrial ROS were the highest in glutamine-deprived HS746T shPHGDH cells. To examine the changes in the transcriptomic landscape including ROS-related genes, the studies performed RNA sequencing analysis on the shPHGDH stable cells. Interestingly, the mitochondrial forms of SHMT and MTHFD2 were significantly up-regulated. Given that folate metabolism is crucial for the cellular NADPH supply, only the mitochondrial isoforms of folate cycle-related genes were up-regulated, highlighting the importance of scavenging mitochondrial ROS upon glutamine starvation. The upregulation of SHMT2 was apparent in HS746T upon glutamine starvation, whereas NCIN87 cells exhibited a reduction in SHMT2 protein levels.

On the basis of the difference in gene expression involved in glycolysis and glutaminolysis between intestinal and SEM-type GC, the study examined the subtype-specific metabolic pathways in GC under nutrient stress, such as glutamine starvation. Although SEM-type GC was expected to be vulnerable to GLS inhibition or glutamine starvation, the study showed that ATF4/CEBPB activates the PHGDH-driven salvage pathway to protect stemness-high cancer cells against mitochondrial ROS in metabolic stress such as glutamine starvation or GLS inhibition.

Without bound to any theory, the development of anticancer drugs targeting metabolic vulnerabilities of cancer would be one therapeutic strategy for cancer treatment. Numerous drugs targeting diverse aspects of nutrient transport and utilization are currently going through clinical trials for various cancers. Furthermore, metabolic alterations have been shown to play pivotal roles in the sensitivity of cancer cells to first-line chemotherapy. Cisplatin-resistant cancer cells have been reported to exhibit dependency on glutamine for nucleotide biosynthesis. Also, cancer cells meet metabolically harsh environment during the course of tumor metastasis. SEM-type GC cells, which are experts in invasion, are expected to have enhanced level of metabolic reprogramming for their survival under the course of metastasis. These findings highlight the potential of therapeutic strategies targeting the metabolic vulnerabilities of chemoresistant cancer cells by identification of key metabolic gene signatures.

SEM-type GC remains one of the most difficult GCs due to drug resistance to standard treatment guidelines. Exploiting metabolic vulnerability would be a promising therapeutic strategy for the treatment of SEM-type GC. Although its gene signature predicted sensitivity to GLS inhibition in SEM-type GC, the SEM-type GC cells showed strong resistance to GLS inhibition or glutamate starvation due to upregulation of the PHGDH-driven salvage pathway. In accordance with our findings, a recent study has also shown that single-drug treatment targeting GLS with CB-839 only exhibited a very limited antitumor effect in glutamine-dependent liver cancer cells. Impressively, CB-839 treatment has been shown to increase metabolic vulnerability in glutamine-dependent liver cancer with antimetabolic drugs, including OXPHOS inhibitors, PPP inhibitors, and PHGDH inhibitors. In our study, combinatorial inhibition of GLS and PHGDH was able to decrease the tumor burden significantly in in vivo model with peritoneal metastasis, which is a debilitating form of metastasis in advanced GC. These results clearly indicate that concomitant inhibition of GLS and PHGDH represents a therapeutic strategy for the treatment of refractory tumors like SEM-type GC.

Most cancer cells proliferate rapidly using glycolysis. Since serine replenishes the 1C unit used in the folate cycle, PHGDH diverts part of the glycolytic flux into the de novo serine synthesis pathway to facilitate the folate cycle and support oligonucleotide synthesis. Accordingly, PHGDH which catalyzes the rate-limiting step of de novo serine synthesis has been widely studied due to its protumorigenic effects. However, little is known about the role of PHGDH in slow-growing cancer cells, such as dormant cancer cells, which do not rely much on glycolysis. In fact, PHGDH is turned “off” in SEM-type GC cells in stress-free conditions. This study reveals that cancer cells with mesenchymal features can turn PHGDH, SHMT2, and MTHFD2 “on” under metabolic stress to control ROS. It has been reported that GLS inhibition in hepatocellular carcinoma attenuated stemness by increasing cellular ROS, which subsequently suppresses the WNT/beta-catenin pathway. However, SEM-type GC cells were able to maintain ROS homeostasis and stemness via activation of a PHGDH-driven mitochondrial folate cycle.

During cancer progression, cancer cells face a metabolically harsh environment. Due to limited nutrient supplies, intermediate metabolites are alternatively consumed for ATP production, putting an emphasis on ROS production rather than ROS elimination. The reported results point to the role of the PHGDH-driven salvage pathway as the central antioxidant system of stemness-high GC cells under glutamine starvation. To explore the reasons for the rapid response detected only in SEM-type GC cell lines upon the same metabolic stress, the studies profiled the chromatin accessibility of two different sub-types of GC cell lines. As stem cells have an open chromatin devoid of heterochromatin as a hallmark, the SEM-type GC cell line showed a high degree of chromatin accessibility and further increase in openness upon glutamine starvation. This key feature in chromatin architecture activates the ATF4/CEBPB-driven gene regulatory network and subsequent gene expression regulatory program. As chromatin architecture has been known to be associated with chemotherapy-induced dormancy and drug resistance, targeting chromatin structure could represent a method to eliminate remnant cancer cells after chemotherapy or to resensitize cancer cells to anticancer drugs. Their high chromatin accessibility allows SEM-type GC cells to activate the salvage pathway, enabling successful defense against oxidative stress and survive under metabolically stressful condition.

In summary, the studies have deciphered in detail the molecular characteristics of SEM-type GC cells including their chromatin structure, gene signatures, and defense mechanism to environmental stress. The result indicates the necessity of combination therapy that simultaneously targets GLS and PHGDH for the treatment of chemoresistant SEM-type GC.

The “Peptide Nucleic Acid (PNA)” referred to an artificially synthesized polymer similar to DNA or RNA, was first introduced in 1991 by Nielsen, Egholm, Berg and Buchardt of the University of Copenhagen, Denmark. While DNA has a phosphate-ribose saccharide backbone, PNA has a repeated N-(2-aminoethyl)-glycine skeleton linked by peptide binding, which greatly increases its binding strength and stability to DNA or RNA, and is used in molecular biology, diagnostic assays, and antisense therapies.

The “aptamer” of the present invention means an oligonucleic acid or peptide molecule.

Based on the amino acid sequence comprising the proteins of the present invention, the Chemical Formulation corresponding to antibodies, PNAs, and aptamers that bind specifically to the protein can be easily produced by ordinary technicians.

The method of measuring the expression level of the gene of the present invention is to perform reverse transcription polymerase reaction (RT-PCR), competitive reverse transcription polymerase reaction (Competitive RT-PCR), real-time reverse transcription polymerase reaction (Real-time RT-PCR), RNase protection assay (RPA; RNase protection assay), Northern blotting, and at least one selected from a group of DNA chips.

The expression level of the gene of the present invention can be measured using a Chemical Formulation that can measure the expression level of the gene. A Chemical Formulation capable of measuring the expression level of the gene may be at least one selected from a group consisting of primers, probes, and antisense nucleotides that bind complementarily to the gene.

The “primer” of the present invention is a fragment recognizing the target gene sequence, comprising a forward and reverse primer pair, but preferably a primer pair providing assay results of specificity and sensitivity. High specificity can be given when the nucleic acid sequence of the primer is inconsistent with the non-target sequence present in the sample, so that the primer amplifies only the target gene sequence containing a complementary primer binding site and does not induce non-specific amplification.

The “probe” of the present invention means a substance that can bind complementarily with the target substance to be detected in the sample, and means a substance that can specifically confirm the presence of the target substance in the sample through the binding. The type of probe is a material commonly used in the industry, but is not limited to, but may preferably be PNA (peptide nucleic acid), LNA (locked nucleic acid), peptide, polypeptide, protein, RNA, or DNA, and most preferably PNA. More specifically, the probe is a biomaterial that includes biological materials derived from or similar to living organisms or manufactured in vitro, e.g., enzymes, proteins, antibodies, microorganisms, animal and plant cells and organs, neurons, DNA, and RNA, DNA includes cDNA, genomic DNA, oligonucleotides, and RNA includes genomic RNA, mRNA, oligonucleotides.

The “Locked nucleic acids” (LNA) of the present invention means nucleic acid analogues comprising a 2′-O, 4′-C methylene bridge [J Weiler, J Hunziker and J Hall Gene Therapy (2006) 13, 496.502]. LNA nucleosides contain common nucleic acid bases from DNA and RNA, and can form base pairs according to the Watson-Crick base pair rule. However, due to the ‘locking’ of the molecules caused by the methylene bridge, the LNA is unable to form the ideal shape in the Watson-Crick bond. When LNA is included in DNA or RNA oligonucleotides, it can more quickly pair with complementary nucleotide chains, increasing the stability of the double helix.

The “antisense nucleotide” of the present invention means an oligomer having a nucleotide sequence and an interunit backbone in which the antisense oligomer is hybridized with the target sequence in RNA by Watson-Crick base pair formation, allowing the formation of mRNA and RNA:oligomer hetero dimers, typically within the target sequence. Oligomers can have exact sequence complementarity or approximate complementarity to the target sequence.

Based on the sequence of the genes of the present invention, the Chemical Formulation corresponding to a primer, probe, etc., which binds complementarily to the gene by a conventional engineer based on the sequence of the genes of the present invention, can be easily manufactured.

The present invention relates to a method of prevention, improvement or treatment of cancer patients, especially EMT (epithelial mesenchymal transition) subtype cancer patients, and in cancer patients with increased expression levels of the GLS (glutaminase) gene or the protein encoded thereby, 1C metabolism is more effectively inhibited when administered in combination with PHGDH, SHMT and MTHFD2 inhibitors, and there is a synergistic effect on the inhibition of cancer cell proliferation in patients with incurable cancer that is difficult to treat due to recurrence, metastasis, and anticancer drug resistance.

Furthermore, by measuring the expression level of the GLS gene or the protein encoded by it, it is possible to increase the success of treatment by providing information about personalized treatment options from an early stage to each patient.

One embodiment of the present invention includes a Chemical Formulation capable of inhibiting the expression of the glutaminase (GLS) gene, or a pharmaceutically acceptable salt thereof; and a drug that can inhibit the expression of any one gene selected from a group consisting of the PHGDH (Phosphoglycerate Dehydrogenase) gene, the SHMT (Serine hydroxymethyltransferase) gene, and the MTHFD2 (Methylenetetrahydrofolate Dehydrogenase (NADP+ Dependent) 2, Methenyltetrahydrofolate Cyclohydrolase) genes, or a pharmaceutical composition for the prevention or treatment of cancer containing a pharmaceutically acceptable salt thereof.

In another embodiment of the present invention, a Chemical Formulation capable of inhibiting the expression of the glutaminase (GLS) gene in an entity requiring administration, or a pharmaceutically acceptable salt thereof; and a method of prevention, improvement or treatment of cancer that includes the administration of a drug that can inhibit the expression of any one gene selected from a group of PHGDH (Phosphoglycerate Dehydrogenase), SHMT (Serine hydroxymethyltransferase) gene, and MTHFD2 (Methylenetetrahydrofolate Dehydrogenase (NADP+ Dependent) 2) gene, MethenyltetrahydrofolateCyclohydrolase, or a composition containing a pharmaceutically acceptable salt thereof as an active ingredient in a pharmacologically effective amount.

Another embodiment of the present invention involves measuring the expression level of the GLS gene or the protein encoded thereby in a biological sample isolated from the object of interest; and if the expression level of the gene or protein measured above is increased, the Chemical Formulation that can inhibit the expression of the following genes; or judging by the concomitant administration of a preparation capable of inhibiting the function of proteins encoded by the following genes; EMT molecules involving subtypes of cancer are related to how to provide information about treatment options.

### EXAMPLES

Hereinafter, the present invention is described in detail by the following embodiment. However, the following embodiment is only an illustration of the present invention, and the content of the present invention is not limited by the following embodiment.

### Example 1: Gastric Transcriptome Analysis

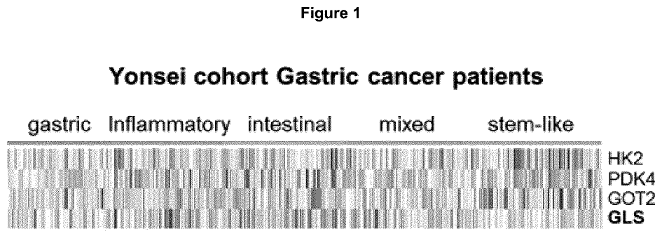

Fresh frozen tumor tissue was obtained from gastric cancer patients who underwent curative intent gastrectomy at Yonsei Cancer Center, and gastric transcriptome analysis data were obtained through the process of matching clinical data. This study was conducted by the Yonsei University College of Medicine Evaluation Board (Institutional Review Board; IRB), and in the case of the above samples, they were collected after written consent from the patient. Gastric cancer patients were divided into 5 subtypes (gastric, inflammatory, intestinal, mixed, and stem-like molecular subtypes) according to the clinically validated classification system, and the genes related to glycolysis and glutaminolysis were analyzed by heatmap according to the usual method, and the results were shown in FIG. 1.

As shown in FIG. 1, transcriptome analysis in Yonsei cohort gastric patients confirmed an increase in the expression level of GLS (glutaminase) gene in patients classified as stem-like subtypes (EMT molecular subtypes) among gastric cancer subtypes as shown in the heat map.

### Example 2: Confirmation of GLS Gene and Protein Expression Levels in Gastric Cancer Cell Line

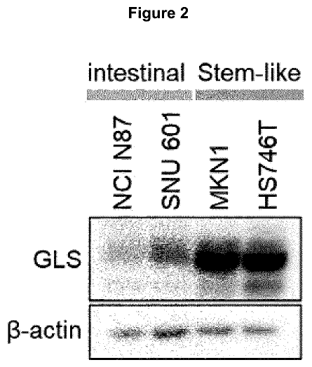

To determine GLS gene and protein expression levels, NCIN87, SNU601, MKN1, and HS746T cell lines were purchased from the Korean cell line bank. Here, in the case of the NCIN87 and SNU601 cell lines, it is a cell line representing the intestinal subtype, and in the case of MKN1 and HS746T cell lines, it corresponds to a cell line representing the stem-like subtype. For these purchased cell lines, RPMI1640 (containing 10% fetal bovine serum (FBS), 2 mM L-glutamine, 100 U/ml penicillin, and 100 μg/ml streptomycin) or DMEM (10% FBS, 2 mM L-glutamine, 100 U/ml penicillin, and 100 μg/ml streptomycin) in a 37° C., 5% CO₂ incubator. At this time, in the case of the above cell lines, all of them were tested for microplasma contamination, and after confirming that the mycoplasma was not contaminated, they were used in the experiment. EBC200 (200 mM NaCl, 50 mM Tris-HCl (pH8.0), 0.5% NP-40) containing a protease inhibitor mixture (genedepot) is placed in each cultured cell line. The process of lysing the cells was carried out. Then, after separating the protein from the lysed cells, the amount of protein was quantified by the BCA analysis method (Pierce), and the same amount of protein was loaded into the SDS-PAGE and electrophoresis was performed, and the SDS-PAGE was transferred to the PVDF membrane (Biorad). After the completed transfer, 5% skim milk (BD difco) was placed in the membrane and blocked for one hour at room temperature, followed by an antibody (abcam) that specifically binds to GLS protein diluted at 1:2000 and ß actin diluted at 1:5000, and incubated at 4° C. for one day. Afterwards, add the secondary antibody diluted in 5% skim milk and incubate for 1 hour, and then use LAS 4000 mini (Fujifilm) to check the expression level of the protein. The results are shown in FIG. 2.

As shown in FIG. 2, compared with NCIN87 and SNU601 cell lines, which correspond to the intestinal subtype, MKN1 and HS746T cell lines, which correspond to the stem-like subtypes, it was found that the expression level of the protein encoded by the GLS gene was significantly increased.

From the above results, it can be seen that the expression level of the protein encoded by the GLS gene is increased, especially in the stem-like subtype compared to other subtypes of gastric cancer.

### Example 3: Genome Analysis Result of Patient-Derived Organoids

Yonsei University College of Medicine Evaluation Board (Institutional Review Board) (IRB) to obtain patient-derived tissues. Then, using the patient-derived tissues, GA326 organoids corresponding to the intestinal subtype and GA077 organoids corresponding to the stem-like subtype were produced.

Specifically, patient tissues classified as enteric or stem-like subtypes according to a clinically validated classification system were obtained and then 40% advanced DMEM/F12 (gibco), 50% Wnt3A cell culture medium (conditioned media), 10% R-spond1 cell culture medium (conditioned media), 1% HEPES (gibco), and 1% GlutaMax (gibco). Organoids were produced using a matrigel to form a 3D structure using a matrigel using a medium containing 0.2% primocin (invivogen), 2% B-27 (invitrogen), 10 mM Nicotinamide (sigma), 1 mM N-Acetylcysteine (sigma), 2 μM A8301, 50 ng/ml mEGF (invitrogen), 100 ng/ml mNoggin (peprotech), 1 nM Gastrin (sigma), 200 ng/ml hFGF10 (peprotech), and 12.5 μM Y-27632 (Enzo).

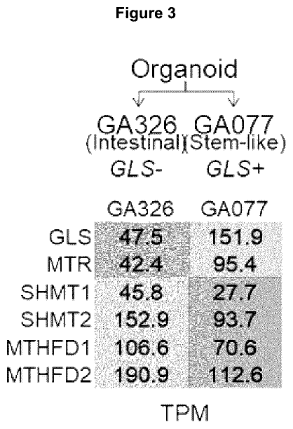

In order to proceed with transcriptome analysis in each of the organoids, the obtained genome analysis data was normalized to TPM, and the transcriptome expression values for the GLS gene and the genes related to monocarbon metabolism, MTR, SHMT1, SHMT2, MTHFD1, and MTHFD2, were identified and shown in FIG. 3.

As shown in FIG. 3, compared to GA326 organoids corresponding to the intestinal subtype, it was confirmed that the expression level of the GLS gene was significantly increased in the GA077 organoids corresponding to the stem-like subtype.

From the above results, it can be seen that the expression level of the GLS gene is particularly increased in the stem-like subtype compared to other gastric cancer subtypes in organoids with high patient-likeness as well as cell lines.

### Example 4: Confirmation of Cell Proliferation of Stem-Like Subtype Cell Lines by GLS Inhibitors

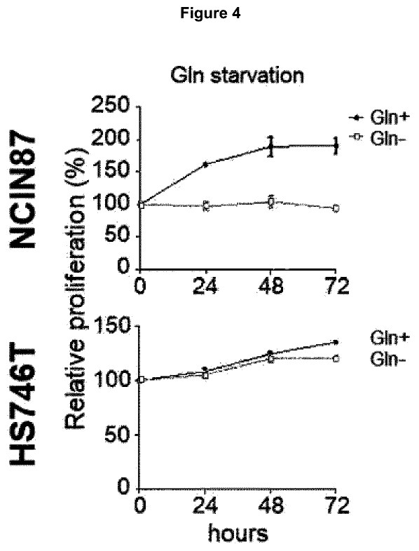

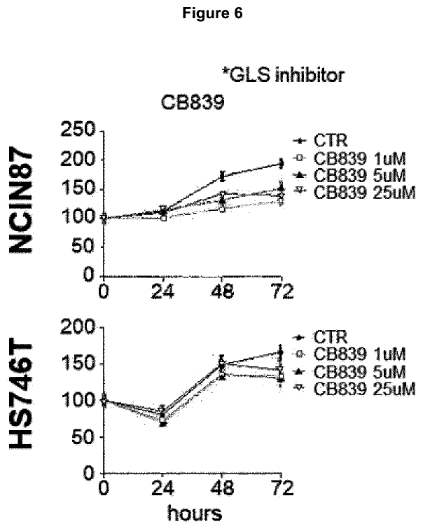

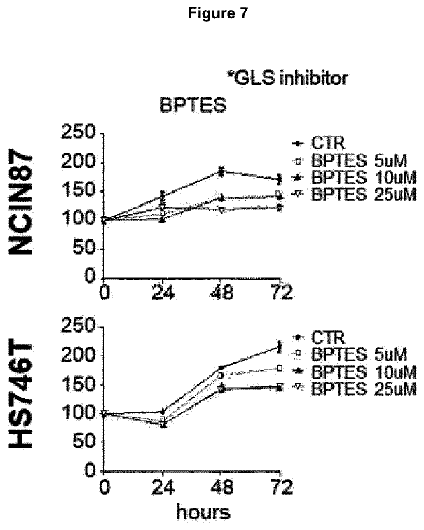

NCIN87, an intestinal subtype cell line, and HS746T, a stem-like subtype cell line, were dispensed in a 96-well plate (black) with 5000 cells each, and replaced with glutamine-deficient culture medium (Gibco) the next day. Then, the cell lines were treated with DON (1 μM, 50 μM, 150 μM; selleckchem, cat no. S8620), CB839 (1 μM, 5 μM, 25 μM; cayman, cat no. S7655) or BPTES (5 μM, 10 μM, 25 μM; MCE, cat no. HY-12683) and the treatment time was 0 hours, and the cell culture medium was discarded at 24, 28 and 72 hours, and stored in an ultra-low temperature freezer at −80° C. After culturing the cells stored in this way at room temperature, 200 μl of cell lysate containing GR dye contained in the cell viability assay kit (CyQUANT cell proliferation assay, Invitrogen) was placed in each well of the cultured cells and incubated at room temperature for 5 min. Afterwards, The intensity of fluorescence was measured at a wavelength of 480 nm and 520 nm excitation using a fluorescence photometer (thermo, varioskan flash 3001), and the measured value was standardized to the number of cells at 0 hour, quantified in %, and the value was shown in FIGS. 4 to 7.

As shown in FIG. 4, cell proliferation was significantly reduced in culture medium (Gln−) without glutamine compared to glutamine-containing culture medium (Gln+) in the intestinal subtype cell line NCIN87, whereas cell proliferation was not reduced with glutamine inclusion in the stem subtype cell line HS746T.

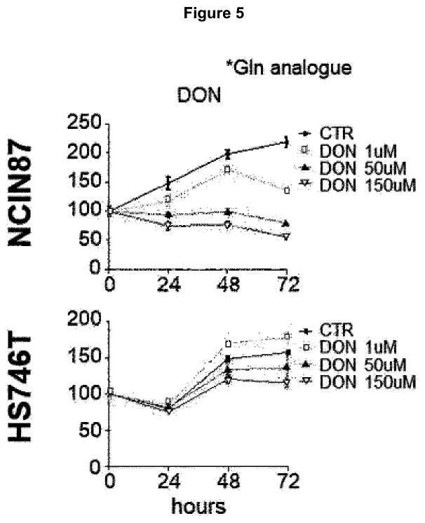

As shown in FIGS. 5 to 7, cell proliferation was inhibited by glutamine analogues (DON) and GLS activity inhibitors (CB839 and BPTES) in the intestinal subtype cell line NCIN87, whereas cell proliferation was not reduced by the drug in the case of the stem-like cell line HS746T.

From the above results, it can be seen that in the case of stem-like subtypes, that is, refractory cancers, cell proliferation is not inhibited by glutamine deficiency or GLS activity inhibitors.

### Example 5: Confirmation of Reduction of Proliferation in Organoids by GLS Inhibitors

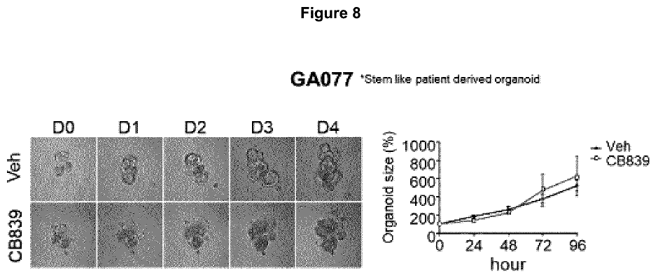

Each of the GA077 organoids produced in Example 3 was treated with 5 μM of CB839 (cayman, cat no. S7655), and the time point of treatment was DO, and it was photographed through an optical microscope (Olympus) on day 1 (D1), day 2 (D2), day 3 (D3), and day 4 (D4), and the longest and shortest diameter passing through the center were measured using image J, and the diameter of the organoid was measured by averaging the two values. Control group (VEH; DMSO-treated group repeated experiment 5 times, and CB839 treatment group repeated experiment 6 times. The organoid diameters measured in this way were converted to μm by microscope size bar, averaged based on the diameter of Day 0, and expressed as a value, which was shown in FIG. 8.

As shown in FIG. 8, the stem-like subtype GA077 organoid was not reduced in size by CB839, which corresponds to the GLS activity inhibitor.

From the above results, it can be seen that in the case of stem-like subtypes, that is, refractory cancers, cell proliferation is not inhibited by GLS activity inhibitors.

### Example 6: Confirmation of Reduction of Proliferation in Stem-Like Subtype Cell Lines by Combination Therapy

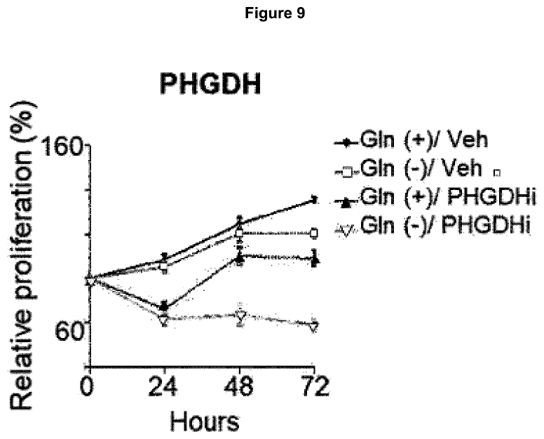

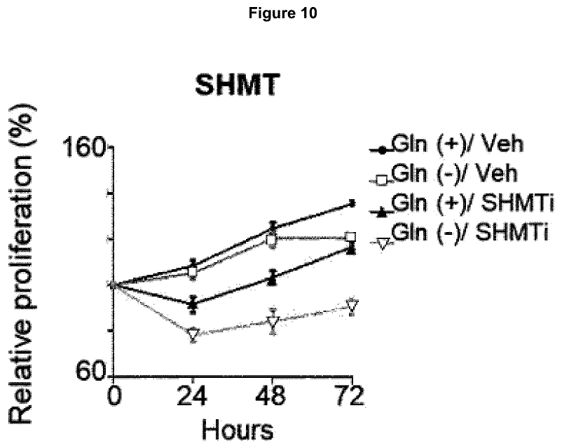

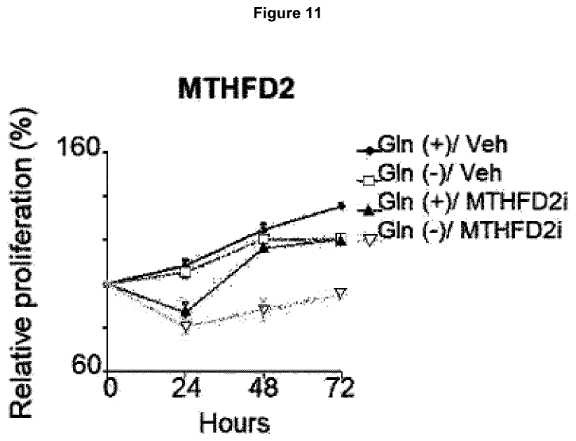

As described in Example 4, after dispensing and incubating the stem-like subtype cell line HS746T, replacing it with a glutamine-deficient culture medium, administering a PHGDH inhibitor (25 μM), SHMT inhibitor (1 μM) or MTHFD2 inhibitor (0.5 μM), and using a cell viability assay kit, the survival rate measured using the cell viability assay kit was shown in FIGS. 9 to 11.

As shown in FIGS. 9 to 11, compared to glutamine deficiency alone (Gln(−)/Veh) and each inhibitor alone (Gln(+)/PHGDHi, Gln(+)/SHMTi, Gln(+)/MTHFD2i), glutamine deficiency treated with PHGDH, SHMT, or MTHFD2 inhibitors alone (Gln(−)/PHGDHi, Gln(−)/SHMTi, Gln(−)/MTHFD2i) found a significant synergistic effect on the survival of the stem-like subtype cell line HS746T.

Based on the above results, in the case of glutamine deficiency and stem-like subtypes that are resistant to GLS inhibitors, i.e., refractory cancers, the combination of PHGDH, SHMT, and MTHFD2 inhibitors can effectively inhibit cell survival.

### Example 7: Confirmation of Reduction in Proliferation in Stem-Like Subtype Organoids by Combination Therapy

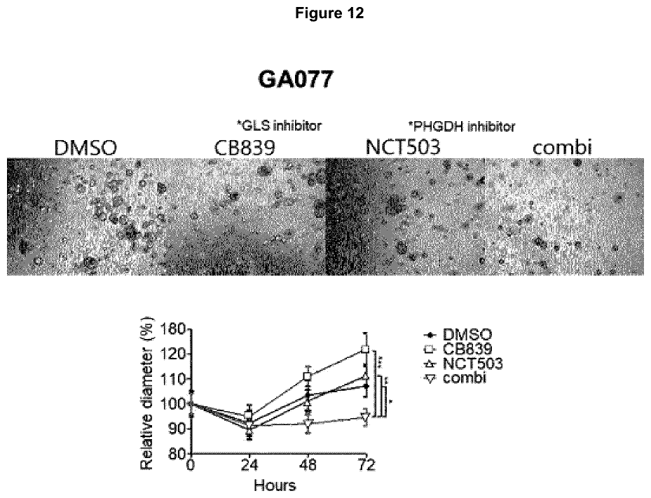

As described in Example 5, GA077, a stem-like subtype organoid, was treated with a GLS inhibitor (CB839, 5 μM), a PHGDH inhibitor (NCT503, 50 μM), or a combination thereof, respectively, and the mean value of the organoid diameter was shown in FIG. 12.

As shown in FIG. 12, the mean value of organoid diameter was significantly reduced when the stem-like subtype organoid GA077 was treated with either a GLS inhibitor (CB839) or a PHGDH inhibitor (NCT503) alone compared to a combination of them (combi).

The results indicates that in the case of stem-like subtypes that are resistant to glutamine deficiency and GLS inhibitors, the combination of PHGDH, SHMT, and MTHFD2 inhibitors can reduce tumor size very effectively.

### Example 8. Single-Nucleus Transcriptome Analysis

Single-Nucleus Transcriptome Analysis Reveals Intratumoral Heterogeneity along with High ATF4/CEBPB Regulon Activity in Cell Subpopulations with High EMT Signature. The efficacy of combinational therapy was evaluated in a more physiological setting. As metastasis is the leading cause of the mortality of GC patients and SEM-type GC cells are more inclined to invade surrounding tissue and metastasize, the study generated the GC peritoneal dissemination model which reflects the nature of SEM-type GC via intraperitoneal injection of HS746T stable cell line with mCherry expression. Peritoneum is a common site of GC metastasis and the GC peritoneal dissemination mouse model successfully mimics metastatic and dissemination tendencies of SEM-type GC. This peritoneal dissemination mouse model was employed to evaluate the actions of GLS inhibitor and PHGDH inhibitor. In accordance with the cytotoxic effect of GLS/PHGDH inhibition in GC cell lines, combinational therapy successfully inhibited tumor growth in the mouse model without a change in mean body weight.

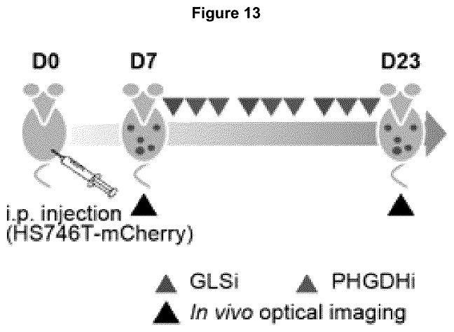

GLS inhibition activates ATF4/CEBPB-mediated transcriptional network in EMT signature-enriched clusters in patient-derived cancer organoids. See FIGS. 13-15.

FIG. 13 is a diagram to explain the process in vivo experiment with vehicle, BPTES (12.5 mg/kg), NCT503 (40 mg/kg), or combination of BPTES and NCT503.

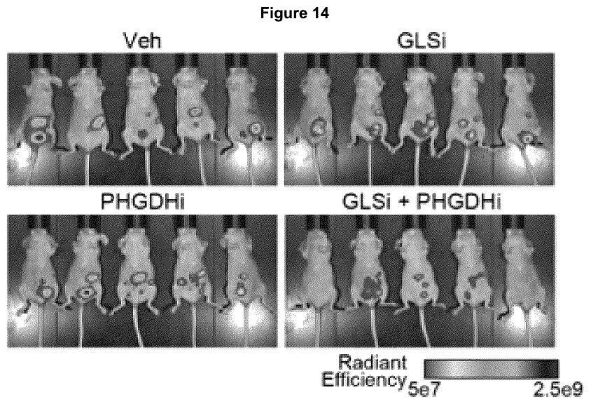

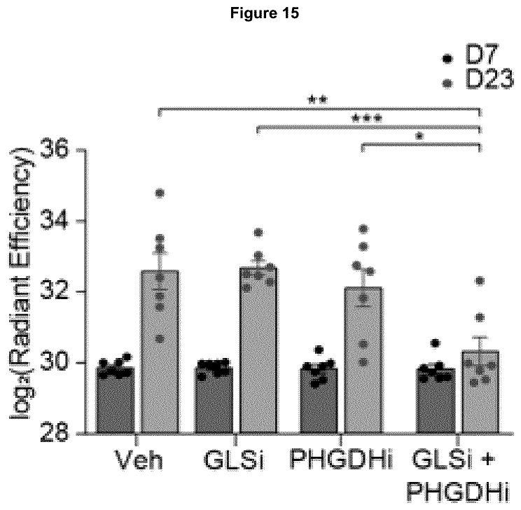

FIG. 14 is a representative picture of mice (n=5) with tumor volume measured via in vivo optical imaging system. Total radiant efficiency (p/sec/cm2/sr/W/cm2) was measured in peritoneal area. FIG. 15 shows that total radiant efficiency was compared in every group (n=7) before (D7) and after (D23) three cycles of BPTES/NCT503 injection.

The above has been described in detail a specific part of the present invention, and it is clear that for a person with ordinary knowledge of the present industry, this specific description is only a desirable embodiment, and therefore the scope of the present invention is not limited. Accordingly, the substantial scope of the present invention will be defined by the attached claims and their equivalents.

The composition according to the present invention can not only inhibit the proliferation of cancer cells, but also has a synergistic effect on the inhibition of the proliferation of cancer cells in patients with incurable cancer that is difficult to treat due to the presence of recurrence, metastasis, and resistance to anticancer drugs, so that cancer can be treated very effectively.

1. A pharmaceutical composition for prevention of treatment of cancer comprising:
a compound or a pharmaceutically acceptable salts thereof for inhibiting expression of a glutaminase (GLS) gene; and
an agent or a pharmaceutically acceptable salt thereof for inhibiting the expression of a gene selected from the group consisting of a Phosphoglycerate Dehydrogenase (PHGDH) gene, a SHMT (Serine hydroxymethyltransferase (SHMT) gene, and an MTHFD2 (Methylenetetrahydrofolate Dehydrogenase (NADP+ Dependent) 2 or MethenyltetrahydrofolateCyclohydrolase) gene, gene.

2. The pharmaceutical composition of claim 1, wherein the agent specifically binds to the mRNA of the gene and is selected from the group consisting of an miRNA, siRNA, shRNA and antisense oligonucleotide.

3. The pharmaceutical composition of claim 1, further comprising an anticancer agent.

4. The pharmaceutical composition of claim 1, wherein the cancer is an epithelial mesenchymal transition (EMT) subtype.

5. The pharmaceutical composition of claim 1, wherein the cancer is gastric cancer, thyroid cancer, parathyroid cancer, ovarian cancer, colorectal cancer, pancreatic cancer, liver cancer, breast cancer, cervical cancer, lung cancer, non-small cell lung cancer, prostate cancer, gallbladder cancer, biliary tract cancer, non-Hodgkin lymphoma, Hodgkin's lymphoma, blood cancer, bladder cancer, head cancer, uterine cancer, rectal cancer, brain tumor, cancer, esophageal cancer, small intestinal cancer, endocrine adenocarcinoma, renal cancer, soft tissue sarcoma, urethral cancer, penis cancer, The pharmaceutical composition of any one selected from the group consisting of renal cell carcinoma, renal pelvic carcinoma, central nerve system (CNS) tumor, primary CNS lymphoma, spinal cord tumor, brain glioma, and pituitary adenoma.

6. A pharmaceutical composition for prevention of treatment of cancer comprising:
a compound or a pharmaceutically acceptable salts thereof for inhibiting a function of a protein expressed by glutaminase (GLS) gene; and
an agent or a pharmaceutically acceptable salt thereof for inhibiting a function of a protein expressed by a gene selected from the group consisting of a PHGDH gene, an SHMT gene, and an MTHFD2.

7. The pharmaceutical composition of claim 6, wherein the compound of the agent is an inverse agonist, or an antagonist, antibody or an aptamer binding to the protein selectively.

8. The pharmaceutical composition of claim 6, further comprising an anticancer agent.

9. The pharmaceutical composition of claim 6, wherein the cancer is an epithelial mesenchymal transition (EMT) subtype.

10. The pharmaceutical composition of claim 6, wherein the cancer is gastric cancer, thyroid cancer, parathyroid cancer, ovarian cancer, colorectal cancer, pancreatic cancer, liver cancer, breast cancer, cervical cancer, lung cancer, non-small cell lung cancer, prostate cancer, gallbladder cancer, biliary tract cancer, non-Hodgkin lymphoma, Hodgkin's lymphoma, blood cancer, bladder cancer, head cancer, uterine cancer, rectal cancer, brain tumor, cancer, esophageal cancer, small intestinal cancer, endocrine adenocarcinoma, renal cancer, soft tissue sarcoma, urethral cancer, penis cancer, The pharmaceutical composition of any one selected from the group consisting of renal cell carcinoma, renal pelvic carcinoma, central nerve system (CNS) tumor, primary CNS lymphoma, spinal cord tumor, brain glioma, and pituitary adenoma.

11. A method for providing information for treating a patient with an epithelial mesenchymal transition (EMT) subtype comprising:
measuring an expression levels of a GLS gene or a protein encoded by it in a biological sample isolated from the object of interest; and
if the expression level of the gene or protein measured above is elevated, determining to concomitantly administrate a composition for inhibiting expression of the following genes and/or an agent capable of inhibiting function of the protein.
